(19) United States
(12) Patent Application Publication
(10) Pub. No.: US 2012/0312318 A1
(43) Pub. Date: Dec. 13, 2012

(54) Two-Phase Developer

(76) Inventors: Marc Krippahl, Monheim (DE); Frank Janssen, Neuss (DE); Armin Wadle, Erkrath (DE); Susanne Hagenow, Hamburg (DE); Sabine Babiel, Moers (DE)

(21) Appl. No.: 13/525,411

(22) Filed: Jun. 18, 2012

Related U.S. Application Data
(63) Continuation of application No. PCT/EP2010/068805, filed on Dec. 3, 2010.

(30) Foreign Application Priority Data
Dec. 16, 2009 (DE) 102009054760.6

(51) Int. Cl. A61K 8/92; A45D 34/00; A61Q 5/10

(52) U.S. Cl. 132/208; 8/406; 132/221

(57) ABSTRACT

Cosmetic preparation for keratin fibers, particularly human hair, having two separate phases, namely, an aqueous phase and a hydrophobic phase. The preparations contain at least one chemical oxidizer and at least one hydrophobic carboxylic acid ester and/or paraffin oil. As oxidizing preparations, the agents are used as a developer preparation for oxidation dyes or lighteners.

### CROSS REFERENCE TO RELATED APPLICATIONS

The present application is a continuation of International Patent Application No. PCT/EP2010/068805 filed 3 Dec. 2010, which claims priority to German Patent Application No. 10 2009 054 760.6, filed 16 Dec. 2009, the contents of both of which are incorporated herein by reference.

### BACKGROUND OF THE INVENTION

The subject matter of the present invention is a cosmetic preparation for keratinic fibers, particularly human hair, wherein the preparation has two phases that are separate from one another, wherein one of the phases represents an aqueous phase and the other phase represents a hydrophobic phase. As oxidizing preparations, these agents are added as a developer preparation for oxidative dyeing or lighteners. Accordingly, a further subject matter of the present invention is a method for changing color of keratinic fibers wherein the inventive agents for the color change are applied onto keratinic fibers.

For the provision of color-changing cosmetic agents, especially for the skin or keratin-containing fibers such as human hair, one skilled in the art is aware of diverse dyeing systems based on dyeing requirements. Oxidation dyes are used for long-lasting, intensive colorations with corresponding authentic characteristics. Dyes of this type usually comprise oxidation dye precursors, developer components and coupler components which together under the influence of oxidizing agents or of atmospheric oxygen form the actual dyes. The oxidation dyes are distinguished by outstanding, long-lasting coloration results. For temporary colorations, usually colorants or toners are used that contain substantive dyes as the coloring component. Many consumers have always wanted, in addition to coloration, to lighten their own hair, as blond hair color is considered attractive and worthwhile from the point of view of fashion. If the intention is to lighten or even bleach substrates, dyes used to color the substrate are removed, mostly oxidatively, by employing appropriate oxidizing agents such as hydrogen peroxide.

In spite of their advantageous dyeing and/or lightening properties, oxidative hair treatment agents in particular present certain disadvantages. Firstly, the use of oxidizing agents damages the hair structure and hair surface. The hair becomes brittle, its elasticity diminishes, and combability worsens. This damage increases with application time. Secondly, oxidative dyes generally need an alkaline pH for dyeing, especially from pH 9.0 to pH 10.5. However, the basic pH associated with the spreading of the external squamosal layer leads to an unpleasant surface feel of the hair and, consequently, to a worse combability when wet and when dry. There is thus a greater need for the consumer to use additional post-treatment agents such as conditioners. Not least however, the hair structure is also negatively affected by external environmental influences. These include mechanical and thermal factors such as combing and use of hair dryers. Meteorological influences, such as wind, rain and UV radiation in sunlight, and additional external stresses, such as chlorinated swimming pool water or perspiration, likewise contribute to damage the hair structure and the hair surface. In order to improve the standard of care of the fibers, it has long been the practice after the color-changing treatment to subject the fibers to a special post-treatment. Here, the hair is treated with special active substances, for example, quaternary ammonium salts or special polymers, usually in the form of a conditioner. Depending on the formulation, combability, hold and volume of the hair are improved and the splitting rate reduced by this treatment. An aggravating factor here is that many of the usual care products and active substances for reducing hair damage are not sufficiently stable under the oxidative, strongly alkaline conditions of a hair treatment agent.

### SUMMARY OF THE INVENTION

Thus, there is still a need for care preparations for the color-changing treatment of fibers. Accordingly, the present invention provides a color-changing agent for reducing the above cited disadvantages of common color-changing agents. In particular, protection against oxidative damage to the hair structure and hair surface should be achieved using the color-changing agent. Care properties of the agent are particularly desirable, so that the user does not need to use additional conditioners and post-treatment agents. Further, the agents should also be suitable for stabilizing care products and active substances that are insufficiently stable under oxidizing conditions, thereby allowing their use in the oxidative hair treatment. It is also desirable for the user if, in addition to care performance, the agents visually emphasize the care.

In an unforeseeable manner, it has now been found that damage to hair can be minimized or a hair care can even be achieved by special two-phase cosmetic, oxidative preparations for use in color-changing agents for keratinic fibers, particularly human hair.

### DETAILED DESCRIPTION OF THE INVENTION

Accordingly, a first subject matter of the present invention is a cosmetic agent for treating keratinic fibers, wherein the agent contains at least two phases that are separated from one another, wherein the first phase (I) is an aqueous phase having at least one chemical oxidizing agent, and wherein the second phase (II) is a hydrophobic phase having at least one liquid carboxylic acid ester of a C₂-C₈ monohydric alkanol with a mono or dicarboxylic acid, and/or at least one paraffin oil.

Keratin-containing or keratinic fibers refer to furs, wool, feathers and particularly human hair. Although the inventive uses are primarily suitable for dyeing and/or lightening keratin-containing fibers, in principle nothing prevents their use in other fields.

A decisive feature of the inventive preparation in this regard is its two-phase nature, with the two phases being immiscible with one another. The two phases are preferably present in two layers one on top of the other with direct contact to one another over a common interface.

In the inventive preparation the weight fraction of phase (I) is at least equal to that of phase (II). There is preferably an excess of phase (I). The weight ratio of phase (I) to phase (II) is preferably 99 to 1 to 50 to 50, more preferably 98 to 2 to 70 to 30, and particularly preferably 95 to 5 to 80 to 20.

According to the present invention, the first phase (I) contains an aqueous or aqueous-alcoholic carrier. For the purposes of the present invention, aqueous-alcoholic carriers refer to water-containing solutions comprising 3 to 70% by weight of a C₁-C₄ alcohol, particularly ethanol or isopropanol, based on total weight of the application mixture. In the context of the invention, an aqueous carrier comprises at least 30 wt %, especially at least 50 wt % water, based on total weight of the application mixture.

The first phase (I) additionally has at least one chemical oxidizing agent. The term “chemical oxidizing agent” here refers to an added extra oxidizing agent and not, for example, an oxidizing agent present in the environment, such as atmospheric oxygen. Hydrogen peroxide is preferably used as the oxidizing agent according to the invention. In this regard, hydrogen peroxide is added either preferably as an aqueous solution or in the form of a solid addition compound of hydrogen peroxide onto inorganic or organic compounds, such as sodium perborate, sodium percarbonate, magnesium percarbonate, sodium percarbamide, polyvinyl pyrrolidone.nH₂O₂ (n is a positive integer greater than 0), urea peroxide and melamine peroxide. Inventively preferred aqueous phases (I) contain aqueous hydrogen peroxide solutions. The concentration of a hydrogen peroxide solution is determined firstly by the legal requirements and secondly by the desired effect. Solutions of 3 wt % to 12 wt % concentration in water are preferably used as the aqueous phase.

One embodiment of the first subject matter of the invention is accordingly one wherein the chemical oxidizing agent of the phase (I) is chosen from hydrogen peroxide and/or one of its solid addition products on inorganic and/or organic compounds.

The inventive preparations preferably comprise hydrogen peroxide. Here, inventive agents for changing the color of keratinic fibers are particularly preferred which comprise 0.5 to 18 wt %, preferably 1 to 15 wt %, more preferably 2.5 to 12 wt % and especially 3 to 9 wt % hydrogen peroxide (calculated as 100% concentrated H₂O₂).

According to the present invention the second phase (II) is of a hydrophobic nature. The inventive hydrophobic phase (II) is immiscible with the aqueous phase (I) containing the oxidizing agent. Hydrophobic phases, also called lipophilic phases, comprise fats that usually have non-polar organic compounds such as hydrocarbon compounds, long-chain triglycerides, silicone oils, esters or ethers as well as perhalogenated compounds.

The hydrophobic phase (II) of the present invention comprises at least one liquid carboxylic acid ester of a C₂-C₈ monohydric alkanol with a mono or dicarboxylic acid, and/or at least one paraffin oil. The term “liquid” in this regard refers to carboxylic acid esters and/or paraffin oils that are liquid at room temperature and under normal pressure. Inventive suitable carboxylic acid esters are those that are very sparingly soluble in water (i.e., a water-solubility of less than 1 g per 1 L water under normal conditions).

The inventive liquid carboxylic acid esters of the hydrophobic phase (II) are derived from C₂-C₈ monohydric alkanols with a mono or dicarboxylic acid.

Exemplary C₂-C₈ monohydric alkanols are ethanol, n-propanol, isopropanol (1-methylethanol), 1-butanol, 2-butanol, 2-methylpropan-2-ol (tert-butanol), 2-methylpropan-1-ol (isobutanol), 1-pentanol, 2-pentanol, 3-pentanol, 3-methylbutan-1-ol (isopentanol), 3-methylbutan-2-ol (siamyl alcohol), 2-methyl-2-butanol, 2,2-dimethylpropan-1-ol (neopentyl alcohol), 1-hexanol, 4-methylpentan-1-ol (isohexanol), 1-heptanol, 1-octanol, 6-methylheptan-1-ol, 3,3-dimethylhexan-1-ol, 3,5-dimethyl-hexan-1-ol, 4,5-dimethylhexan-1-ol, 3-methylheptan-1-ol and 5-methylheptan-1-ol (isooctanols) and 2-ethylhexan-1-ol (ethyihexyl alcohol). Exemplary suitable monocarboxylic acids are saturated fatty acids such as decanoic acid, dodecanoic acid (lauric acid), tetradecanoic acid (myristic acid), hexadecanoic acid (palmitic acid), octadecanoic acid (stearic acid) and 16-methylheptadecanoic acid (isostearic acid), unsaturated fatty acids like palmitoleic acid (C16:1; 9Z), oleic acid (C18:1; 9Z), elaidinoic acid (C18:1; 9E), eicosenoic acid (gondoic acid; C20:1; 11Z), linoleic acid (C18:2; 9Z, 12Z), γ-(gamma)-linolenic acid (C18:3; 6Z, 9Z, 12Z), α-(alpha)-linolenic acid (C18:3; 9Z, 12Z, 15Z), α-elaeostearic acid (C18:3; 9Z, 11E, 13E) and arachidic acid (C20:4; 5Z, 8Z, 11Z, 14Z) as well as natural fatty acid fractions like fatty acids from lanolin (lanolates), coco fat (cocoates) and tallow fat (tallowates). Exemplary suitable dicarboxylic acids are saturated dicarboxylic acids with 4 to 10 carbon atoms, particularly succinic acid (ethane-1,2-dicarboxylic acid), glutaric acid (propane-1,3-dicarboxylic acid), adipic acid (butane-1,4-dicarboxylic acid) and sebacic acid (octane-1,8-dicarboxylic acid).

In this regard, those carboxylic acid esters having a total number of carbons from 12 to 22 carbon atoms are preferred. Preferred compounds are ethyl laurate, propyl laurate, isopropyl laurate (ipl), butyl laurate, hexyl laurate, ethylhexyl laurate, ethyl myristate, propyl myristate, isopropyl myristate (ipm), butyl myristate, hexyl myristate, ethylhexyl myristate, ethyl palmitate, propyl palmitate, isopropyl palmitate (ipp), butyl palmitate, hexyl palmitate, ethyl stearate, propyl stearate, isopropyl stearate (ips), butyl stearate, ethyl isostearate, propyl isostearate, isopropyl isostearate (ipis), butyl isostearate, ethyl oleate, propyl oleate, isopropyl oleate (ipo), butyl oleate, diethyl succinate, dipropyl succinate, diisopropyl succinate, dibutyl succinate, dihexyl succinate, diethylhexyl succinate, diethyl glutarate, dipropyl glutarate, diisopropyl glutarate, dibutyl glutarate, dihexyl glutarate, diethyl adipate, dipropyl adipate, diisopropyl adipate, dibutyl adipate, dihexyl adipate, diethyl sebacinate, dipropyl sebacinate, diisopropyl sebacinate, dibutyl sebacinate and dihexyl sebacinat.

A further embodiment of the first subject matter of the invention is one wherein the carboxylic acid ester of the hydrophobic phase (II) is an ester of C₃-C₄ monohydric alkanol with a mono or dicarboxylic acid.

Isopropanol and butan-1-ol are particularly suitable as the C₃-C₄ monohydric alkanol. Particularly preferred carboxylic acid esters are therefore chosen from isopropyl laurate (IPL), butyl laurate, isopropyl myristate (IPM), butyl myristate, isopropyl palmitate (IPP), butyl palmitate, isopropyl stearate (IPS), butyl stearate, isopropyl isostearate (IPIS), butyl isostearate, isopropyl oleate (IPO), butyl oleate, diisopropyl succinate, dibutyl succinate, diisopropyl glutarate, dibutyl glutarate, diisopropyl adipate, dibutyl adipate, diisopropyl sebacinate and dibutyl sebacinate. Isopropyl myristate (IPM), isopropyl palmitate (IPP) and dibutyl adipate are particularly preferred. These compounds are commercially available and are marketed inter alia under the trade names Croda-mol IPM, Stepan IPM or Lexol IPM NF, or Rilanit IPP or Nikkol IPP, as well as Cetiol B (dibutyl adipate).

A further embodiment of the first subject matter of the invention is accordingly one wherein the carboxylic acid ester of the hydrophobic phase (II) is chosen from isopropyl palmitate, isopropyl myristate and dibutyl adipate.

Paraffin oils are not water-soluble and are therefore suitable as the hydrophobic phase (II) in the inventive two-phase agents. Paraffin oils of the hydrophobic phase refers to mixtures of saturated, aliphatic hydrocarbons that are liquid at room temperature. Accordingly, inventively preferred agents have at least one paraffin oil as the hydrophobic phase (II).

In order to improve separation of the hydrophilic phase (I) and hydrophobic phase (II) and to reduce the tendency to form a stable emulsion, it is preferred that the agent comprise only a minor amount of surface active substances. In the context of the invention, emulsifiers and surfactants are considered as surface active substances. Surface active substances have hydrophobic and hydrophilic structural features, thereby enabling the phases when mixed together to form micelles and stable emulsions. As the present invention contains no emulsions but rather comprises phases that are separate from one another, it has proved particularly advantageous for the agent to comprise a total weight of non-ionic, anionic, zwitterionic and/or amphoteric surfactants and/or emulsifiers of less than 5 wt %, preferably less than 1 wt %, based on total weight of the ready-for-use agent. Agents that are free of surface active substances are particularly advantageous.

In the context of the invention, anionic surfactants refer to all anionic surface-active materials suitable for use on the human body. They have a water-solubilizing anionic group such as a carboxylate, sulfate, sulfonate or phosphate group and a lipophilic alkyl group containing about 8 to 30 carbon atoms. In addition, the molecule may comprise glycol or polyglycol ether groups, ester, ether and amide groups as well as hydroxyl groups. Exemplary suitable anionic surfactants are, each in the form of the sodium, potassium and ammonium, as well as the mono, di and trialkanolammonium salts containing 2 to 4 carbon atoms in the alkanol group, linear and branched fatty acids with 8 to 30 carbon atoms (soaps); ether carboxylic acids, particularly of the formula RO(CH₂CH₂O)ₓCH₂COOH wherein R is a linear alkyl group with 8 to 30 carbon atoms and x is 0 or 1 to 16; acylsarcosides; acyltaurides; acylisethionates; sulfosuccinic acid mono and dialkyl esters, as well as sulfosuccinic acid monoalkyl polyoxyethyl esters; linear alkane sulfonates; linear a-olefin sulfonates; sulfonates of unsaturated fatty acids; a-sulfofatty acid methyl esters of fatty acids; alkyl sulfates and alkyl ether sulfates, particularly of the Formula RO(CH₂CH₂O)ₓSO₃H wherein R is a linear alkyl group with 8 to 30 carbon atoms and x is 0 or a number from 1 to 12; mixtures of surface-active hydroxysulfonates; sulfated hydroxyalkyl polyethylene glycol ethers and/or hydroxyalkylene propylene glycol ethers; esters of tartaric acid and citric acid with alcohols; alkyl and/or alkenyl ether phosphates of the Formula RO(C₂H₄O)ₓP(═O)(OH)(OR′) wherein R is an aliphatic, optionally unsaturated hydrocarbon group with 8 to 30 carbon atoms, R′ is hydrogen, a group (CH₂CH₂O)_{y}R, and x and y independently of one another are a number from 1 to 10; sulfated fatty acid alkylene glycol esters of the Formula RC(O)O(alkO)ₙSO₃H wherein R is a linear or branched, aliphatic, saturated and/or unsaturated alkyl group with 6 to 22 carbon atoms, alk is CH₂CH₂, CHCH₃CH₂ and/or CH₂CHCH₃, and n is a number from 0.5 to 5; as well as monoglyceride sulfates and monoglyceride ether sulfates.

Zwitterionic surfactants are those surface-active compounds having at least one quaternary ammonium group and at least one carboxylate, sulfonate or sulfate group in the molecule. Exemplary suitable zwitterionic surfactants are betaines such as the N-alkyl-N,N-dimethylammonium glycinates, for example, cocoalkyl dimethylammonium glycinate, N-acylaminopropyl-N, N-dimethylammonium glycinates, for example, cocoacylaminopropyl-dimethylammonium glycinate, and 2-alkyl-3-carboxymethyl-3-hydroxyethyl imidazolines with 8 to 18 carbon atoms in each of the alkyl or acyl groups, as well as cocoacylaminoethyl hydroxyethylcarboxymethyl glycinate. A preferred zwitterionic surfactant is the fatty acid amide derivative known under the INCI name Cocamidopropyl Betaine.

Ampholytic surfactants include such surface-active compounds that, apart from a C₈-C₂₄ alkyl or acyl group, have at least one free amino group and at least one —COOH or —SO₃H group in the molecule and are able to form internal salts. Common amphoteric surfactants are N-alkylglycines, N-alkyl propionic acids, N-alkylamino butyric acids, N-alkylimino dipropionic acids, N-hydroxyethyl-N-al kylamidopropylglycines, N-alkyltaurines, N-alkylsarcosines, 2-alkylamino propionic acids and alkylamino acetic acids, each with about 8 to 24 carbon atoms in the alkyl group. Particularly preferred amphoteric surfactants are N-cocoalkylamino propionate, cocoacylaminoethylamino propionate and C₁₂-C₁₈ acyl sarcosine.

Non-ionic surfactants and emulsifiers comprise, for example, a polyol group, a polyalkylene glycol ether group or a combination of polyol and polyglycol ether groups as the hydrophilic group. Exemplary compounds of this type are addition products of 1 to 50 moles ethylene oxide and/or 0 to 5 moles propylene oxide to linear and branched fatty alcohols containing 8 to 30 carbon atoms, to fatty acids containing 8 to 30 carbon atoms and to alkyl phenols containing 8 to 15 carbon atoms in the alkyl group; methyl or C₂-C₆ alkyl group end blocked addition products of 1 to 50 moles ethylene oxide and/or 0 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group, such as the commercially available types Dehydol® LS, Dehydol® LT (Cognis); polyglycerin esters and alkoxylated polyglycerin esters, such as poly(3)glycerine diisostearate (commercial product: Lameform TGI (Henkel)) and poly(2)glycerin polyhydroxystearate (commercial product: Dehymuls PGPH (Henkel)); polyol esters of fatty acids, such as the commercial product Hydagen® HSP (Cognis) or Sovermol types (Cognis); higher alkoxylated, propoxylated and in particular ethoxylated mono, di and triglycerides with alkoxylation degrees of greater than 5, such as glycerin monolaurate+20 ethylene oxide, and glycerin monostearate+20 ethylene oxide; amine oxides; hydroxy mixed ethers; sorbitol esters of fatty acids and addition products of ethylene oxide to sorbitol esters of fatty acids such as the polysorbates and sorbitol monolaurate+20 moles ethylene oxide (EO); sugar esters of fatty acids and addition products of ethylene oxide to sugar esters of fatty acids; addition products of ethylene oxide to fatty acid alkanolamides and fatty amines; fatty acid N-alkylglucamides; alkylphenols and alkylphenol alkoxylates with 6 to 21, particularly 6 to 15 carbon atoms in the alkyl chain and 5 to 30 ethylene oxide and/or propylene oxide units; alkyl polyglycosides corresponding to the general formula RO—(Z)ₓ wherein R is alkyl, Z is sugar and x is the number of sugar units. In the context of the invention, non-ionic emulsifiers further include the polymerization products of ethylene oxide and propylene oxide on saturated or unsaturated fatty alcohols; fatty acid esters of polyhydric alcohols with saturated or unsaturated fatty acids; alkyl esters of saturated or unsaturated fatty acids or alkylphenols and their alkoxylates; particularly ethylene glycol ethers of fatty alcohols; mixed ethylene glycol ethers and propylene glycol ethers with fatty alcohols; fatty acid esters on sorbitol as well as polyethylene glycol; esters of non-hydroxylated C₆-C₃₀ alkylmonocarboxylic acids with polyethylene glycol; and addition products of alkylphenols to ethylene oxide and/or propylene oxide.

Furthermore, it can be advantageous to additionally add electrolyte to the agent for the separation of the hydrophilic and hydrophobic phase in the agent. Electrolytes typically refer to charged, ionic inorganic and organic compounds having no or only a very slightly pronounced hydrophobic fraction. Preferred electrolytes are highly soluble salts, particularly alkali metal and alkaline earth metal salts of mineral acids and organic acids. Examples of these are sodium chloride, sodium sulfate, sodium hydrogen sulfate, sodium carbonate, sodium hydrogen carbonate, sodium citrate, magnesium chloride, magnesium sulfate, magnesium carbonate and magnesium hydrogen carbonate.

Oil-soluble ingredients in the inventive agent mainly accumulate in the hydrophobic phase (II) and therefore do not come into contact with the oxidizing agent-containing phase (I). This is particularly advantageous for stabilizing oxidatively less stable care substances in the agent. Such preferred care substances are consequently oil-soluble care substances, oil-soluble vitamins and triglycerides, particularly vegetal and those having one or more unsaturated carbon-carbon bonds. In order to visually accentuate the two-phase nature, it can likewise be sensible for the hydrophobic phase (II) to contain oil-soluble dyes.

Accordingly, a particular embodiment of the present invention is one wherein the agent additionally contains at least one predominantly oil-soluble component chosen from oil-soluble dyes, oil-soluble care substances, oil-soluble vitamins and triglycerides.

Inventively designated predominantly oil-soluble compounds are those with a water-solubility of less than 1 g per 1 L water under normal conditions, but which are highly soluble in non-polar compounds (i.e., >10 g/kg solvent medium).

Exemplary oil-soluble care substances are cosmetically active terpenes and terpenoids, such as bisabolol, and ubiquinones such as coenzyme Q-10.

Oil-soluble vitamins are in particular compounds known collectively as vitamin A, vitamin D, vitamin E and vitamin K. An inventively preferred agent therefore comprises at least one oil-soluble vitamin chosen from vitamin A, vitamin D, vitamin E and/or vitamin K as well as vitamin P. Here, vitamin A includes retinoids, particularly all-trans-retinol. Vitamin D, also called calciferols, includes 7,8-didehydrosterol derivatives, particularly the compounds named cholecalciferol (vitamin D₃, calciol), ergo-calciferol (vitamin D₂, ercalciol), 7,8-didehydrocholesterol (provitamin D₃, procalciol, prochole-calciferol) and ergosterol (provitamin D₂). Additional, usable vitamin D analogs are calcidiol (25-hydroxycholecalciferol), calcitriol, Hydroxycalcidiol and vitamin D₁ (ergocalciferol and lumilsterol). Vitamin E is the collective term for tocopherols and includes in particular the chemical compounds a-tocopherol, β-tocopherol, γ-tocopherol, δ-tocopherol and α-tocotrienol, β-tocotrienol, γ-tocotrienol and δ-tocotrienol. Vitamin K is the collective term for various compounds with vitamin K activity, which are derived from 2-methyl-1,4-naphthoquinone (vitamin K₃). Preferred representatives are vitamin K₁₍₂₀₎ (2-methyl-3-phytyl-1,4-naphthoquinone), phylloquinone (abbreviation: K),], vitamin K₂₍₃₅₎ (3-all-trans-farnesylgeranylgeranyl-2-methyl-1,4-naphthoquinone), vitamin K₃ (2-methyl-1,4-naphthoquinone, menadione, menaphthone) as well as the derived analogs vitamin K₄ (2-methyl-1,4-naphthaline diol), vitamin K₅ (4-amino-2-methyl-1-naphthol), vitamin K₅ (2-methyl-1,4-naphthalinediamine) and vitamin K₇ (4-amino-3-methyl-1-naphthol). Vitamin P concerns a collective name for rutine, in particular bioflavonoids like troxerutine (vitamin P₄) and hesperidine.

Triglycerides are the collective term for esters of glycerin, which represent the main constituent of oils of natural origin. Inventively particularly preferred triglycerides are those having at least one ester of an unsaturated fatty acid. Preferred unsaturated fatty acids are oleic acid, linoleic acid and linolenic acid. Moreover, vegetal oils can preferably be used as the triglycerides, particularly those having a positive influence on the hair surface. In this regard, particularly suitable triglycerides are in particular oils obtained from the seeds of Moringa pterygosperma (moringa oil) or from the kernals of Argania Spinosa (argan oil). These oils are marketed, for example, under the name Lipofructyl® ARGAN LS 9779 or Lipofructyl® MO LS 9305 by the Cognis Company.

One embodiment of the first subject matter of the invention is consequently one wherein the hydrophobic phase (II) additionally has at least one oil chosen from oils from the seeds of Moringa pterygosperma (moringa oil) and/or from the kernals of Argania Spinosa (argan oil).

The predominantly oil-soluble components are preferably incorporated in a total weight of 0.001 to 10 wt %, particularly 0.01 to 5 wt %, based on total weight of the hydrophobic phase (II).

The inventive agents are preferably used for changing the color of keratinic fibers. For this, the inventive two-phase agent (M1) is blended with another agent (M2) having at least one color-changing component, and the resulting, ready-for-use preparation is added onto the keratinic fibers.

As the color-changing component in agent (M2), additional bleach boosters act as the lightening agent by boosting the action of the oxidizing agent of phase (I) of the two-phase agent, as well as chromophoric components.

Accordingly, in one embodiment the inventive agent (M2) comprises an additional bleach booster. In the context of this invention, carbonic acid derivatives, alkyl carbonates and alkyl carbamates, as well as silyl carbonates and silyl carbamates, can be employed as additional bleach boosters, compounds that under perhydrolysis conditions afford aliphatic peroxycarboxylic acids and/or substituted perbenzoic acid.

The bleach booster is preferably chosen from ammonium peroxydisulfate, alkali metal peroxydisulfates, ammonium peroxymonosulfate, alkali metal hydrogen peroxymonosulfates, alkali metal peroxydiphosphates and alkaline earth metal peroxides. Particularly preferred bleach boosters are ammonium peroxydisulfate, potassium peroxydisulfate, sodium peroxydisulfate, potassium hydrogen peroxymonosulfate, potassium peroxydiphosphate, magnesium peroxide and barium peroxide. Inventively particularly preferred agents comprise at least one inorganic salt, selected from peroxymonosulfates and/or peroxydisulfates as the bleach booster. Moreover, it has been determined that agents according to the invention should particularly preferably comprise at least two different peroxydisulfates. In this regard, preferred peroxydisulfate salts are combinations of ammonium peroxydisulfate and potassium peroxydisulfate and/or sodium peroxydisulfate. The ready for use agent preferably contains peroxy compounds in an amount of 0.1 to 25 wt %, particularly 0.5 to 15 wt %, based on total weight of the ready for use agent.

The persulfate salts or peroxydisulfate salts are generally anhydrous and added in the form of an optionally dedusted powder, paste or in the form of a compressed molded body. The anhydrous agents (M2) can comprise an additional bleach booster instead of and/or in addition to the solid peroxy compounds.

Bleach activators that can be used are compounds which, under perhydrolysis conditions, yield aliphatic peroxycarboxylic acids having preferably 1 to 10 carbon atoms, particularly 2 to 4 carbon atoms, and/or optionally substituted perbenzoic acid. Substances which carry O-acyl and/or N-acyl groups of said number of carbon atoms and/or optionally substituted benzoyl groups are suitable. Preference is given to polyacylated alkylenediamines, particularly tetraacetyl ethylenediamine (TAED), acylated triazine derivatives, particularly 1,5-diacetyl-2,4-dioxohexahydro-1,3,5-triazine (DADHT), acylated glycolurils, particularly tetraacetyl glycoluril (TAGU), N-acylimides, particularly N-nonanoyl succinimide (NOSI), acylated phenol sulfonates, particularly n-nonanoyl- or isononanoyloxybenzene sulfonate (n- or iso-NOBS), carboxylic acid anhydrides, particularly phthalic anhydride, acylated polyhydric alcohols, particularly triacetin, ethylene glycol diacetate and 2,5-diacetoxy-2,5-dihydrofuran.

Carbonate salts or hydrogen carbonate salts can be inventively preferably used as bleach boosters of the carbonic acid derivative type. They are preferably chosen from carbonate salts or hydrogen carbonate salts of ammonium, alkali metals (especially sodium and potassium) as well as of alkaline earth metals (especially magnesium and calcium). Particularly preferred carbonate or hydrogen carbonate salts are ammonium hydrogen carbonate, ammonium carbonate, sodium hydrogen carbonate, sodium carbonate, potassium hydrogen carbonate, potassium carbonate, magnesium carbonate and calcium carbonate. These particularly preferred salts can be used as bleach boosters singly or in mixtures of at least two representatives.

Although in principle there is no limitation regarding the formulation of the additional agent (M2), it is inventively preferred when agent (M2) is an anhydrous formulation. In the present invention, anhydrous means a water content, based on agent (M2), of 5 wt % or less, especially 2 wt % or less. Blonding preparations having 0.1 wt % or less water can be inventively quite particularly preferred. The agent (M2) is preferably formulated as a powder or an anhydrous paste.

Bleach boosters of the alkyl carbonate and alkyl carbamate type as well as silyl carbonate and silyl carbamate can be incorporated in the anhydrous compositions as the bleach booster and are characterized by compounds according to Formula (BV)

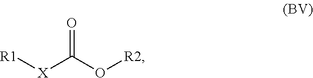

wherein

R1 is a saturated or unsaturated, straight chain, branched, or cyclic, substituted or unsubstituted hydrocarbon group, or a substituted or unsubstituted aryl group or a substituted or unsubstituted heterocycle,

X is O or NR3, wherein R3 is a hydrogen atom, a saturated or unsaturated, straight chain, branched, or cyclic, substituted or unsubstituted hydrocarbon group, or a substituted or unsubstituted silyl group, or a substituted or unsubstituted aryl group, or a substituted or unsubstituted heterocycle, and

R2 is a hydrogen atom, an alkali metal atom, particularly sodium, or a group SiR₃ wherein the R groups independently of each other are a hydrogen atom, a saturated or unsaturated, straight chain, branched, or cyclic, substituted or unsubstituted hydrocarbon group, or a trialkylsilyl group, preferably a trimethylsilyl group, or a substituted or unsubstituted aryl group, or a substituted or unsubstituted heterocycle, or a halide, a substituted or unsubstituted hydroxyl or amino group.

Inventively particularly preferred compositions are those wherein the group R1 in Formula (BV) is chosen from methyl, ethyl, n-propyl, i-propyl, n-butyl, i-butyl, t-butyl as well as hydroxymethyl and hydroxyethyl. Preferred R2 and R3 groups in the Formula (BV) are hydrogen, substituted or unsubstituted, straight chain or branched alkyl groups as well as trialkylsilyl groups. Among these, hydrogen, methyl, ethyl, t-butyl and trimethylsilyl groups are preferred.

Compositions according to the invention can preferably contain at least one compound chosen from acetic acid, lactic acid, tartaric acid, citric acid, salicylic acid and ortho-phthalic acid as an additional bleach booster.

In another preferred embodiment the agent (M2) can have at least one cationic pyridinium derivative as the bleach booster. Preferred compounds are 4-acylpyridinium derivatives and 2-acylpyridinium derivatives. In this regard, 2-acetyl-1-methylpyridinium p-toluenesulfonate and 4-acetyl-1-methylpyridinium p-toluenesulfonate are particularly preferred. Additionally preferred cationic pyridinium derivatives are cationic 3,4-dihydroisoquinolinium derivatives. N-methyl-3,4-dihydroisoquinolinium p-toluenesulfonate is particularly preferred.

The bleach boosters employed in addition to, or instead of, peroxy compounds are present in cosmetic agents according to the invention preferably in amounts of 0.05 to 10% by weight, particularly 0.2 to 5% by weight, based on total weight of the ready-for-use agent.

In order to further increase the lightening power, at least one optionally hydrated SiO₂ compound can be added as the bleach booster to the agent. Although small amounts of optionally hydrated SiO₂ compounds already increase the lightening power, it may be preferred to use optionally hydrated SiO₂ compounds in amounts of 0.05% to 15% by weight, more preferably 0.15% to 10% by weight, and quite preferably 0.2% to 5% by weight, based on the anhydrous agent according to the invention. Here, the quantities reflect the content of the SiO₂ compounds (without their water content) in the agents. Preferred optionally hydrated SiO₂ compounds are silicic acids, their oligomers and polymers, and their salts. Optionally hydrated SiO₂ compounds can be present in various forms. According to the invention, SiO₂ compounds are preferably used in the form of silica gels or particularly preferably as a water-glass. According to the invention, quite particular preference is given to water-glasses formed from a silicate of the formula (SiO₂)ₙ(Na₂O)ₘ(K₂O)ₚ, where n is a positive rational number and m and p, independently of one another, are a positive rational number or are 0, with the provisos that at least one of the parameters m or p is different from 0 and the ratio between n and the sum of m and p is from 1:4 to 4:1. Metasilicates can preferably be employed, particularly those in the above Formula having the ratio between n and the sum of m and p being 1 or less and which can be considered as chain-like polymeric structures of the anion [SiO₃]²⁻. In this regard, sodium metasilicate of the Formula [Na₂SiO₃]ₓ is particularly preferred.

In another embodiment, the agent (M2) comprises chromophoric components as the color-changing component. Consequently, the inventive agents can additionally comprise at least one chromophoric component preferably chosen from at least one oxidation dye precursor and/or from at least one substantive dye.

Therefore, inventively preferred agents for changing the color of keratinic fibers comprise at least one oxidation dye precursor. The inventive lighteners comprise at least one oxidation dye precursor of the developer type (developer component) as the oxidation dye precursor, preferably in combination with at least one oxidation dye precursor of the coupler type.

Preferred oxidation dye precursors of the developer type are p-phenylenediamine derivatives. Preferred p-phenylenediamines are chosen from one or more compounds of p-phenylenediamine, p-toluylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediam ine, N,N-dimethyl-p-phenylenediamine, N,N-diethyl-p-phenylenediamine, N,N-dipropyl-p-phenylenediamine, 4-amino-3-methyl-(N,N-diethyl)-aniline, N,N-bis-(2-hydroxyethyl)-p-phenylenediamine, 4-N,N-bis-(2-hydroxyethyl)-ami no-2-methylaniline, 4-N,N-bis-(2-hydroxyethyl)-amino-2-chloroaniline, 2-(2-hydroxyethyl)-p-phenylenediamine, 2-(1,2-dihydroxyethyl)-p-phenylenediamine, 2-fluoro-p-phenylenediamine, 2-isopropyl-p-phenylenediamine, N-(2-hydroxypropyl)-p-phenylenediamine, 2-hydroxymethyl-p-phenylenediamine, N,N-d imethyl-3-methyl-p-phenylenedi amine, N-ethyl-N-2-hydroxyethyl-p-phenylenediamine, N-(2,3-dihydroxypropyl)-p-phenylenediamine, N-(4′-aminophenyl)-p-phenylenediamine, N-phenyl-p-phenylenediamine, 2-(2-hydroxyethyloxy)-p-phenylenediamine, 2-methoxymethyl-p-phenylenediamine, 2-(2-acetylaminoethyloxy)-p-phenylenediamine, N-(2-methoxyethyl)-p-phenylenediamine, N-(4-amino-3-methylphenyl)-N-[3-(1H-imidazol-1-yl)propyl]amine, 5,8-diaminobenzo-1,4-dioxane as well as their physiologically acceptable salts. Inventively particularly preferred p-phenylenediamine derivatives are selected from at least one compound of the group p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, N-(4-amino-3-methylphenyl)-N-[3-(1H-imidazol-1-yl)propyl]amine, 2-methoxymethyl-p-phenylenediamine, as well as the physiologically acceptable salts of these compounds.

According to the invention, it may also be preferred to use as the developer component compounds having at least two aromatic nuclei that are substituted by amino and/or hydroxyl groups. Preferred binuclear developer components are especially chosen from at least one of: N,N′-bis-(2-hydroxyethyl)-N,N′-bis-(4′-aminophenyl)-1,3-diaminopropan-2-ol, N,N′-bis-(2-hydroxyethyl)-N,N′-bis-(4′-aminophenyl)-ethylendiamine, N,N′-bis-(4′-aminophenyl)tetramethylenediamine, N,N′-bis-(2-hydroxyethyl)-N,N′-bis-(4′-aminophenyl)-tetramethylenediamine, N,N′-bis-(4-(methylamino)phenyl)tetramethylenediamine, N,N′-Diethyl-N,N′-bis-(4′-amino-3′-methylphenyl)ethylenediamine, bis-(2-hydroxy-5-aminophenyl)-methane, N,N′-bis-(4′-aminophenyl)-1,4-diazacycloheptane, N,N′-bis-(2-hydroxy-5-aminobenzyl)-piperazine, N-(4′-aminophenyl)-p-phenylendiamine and 1,10-bis-(2′,5′-diaminophenyl)-1,4,7,10-tetraoxadecane as well as their physiologically acceptable salts. Quite particularly preferred binuclear developer components are selected from among N,N′-bis-(2-hydroxyethyl)-N,N′-bis-(4-aminophenyl)-1,3-diamino-2-propanol, bis-(2-hydroxy-5-aminophenyl)methane, 1,3-bis-(2,5-diaminophenoxy)-2-propanol, N,N′-bis(4-aminophenyl)-1,4-diazacycloheptane, 1,10-bis-(2,5-diaminophenyl)-1,4,7,10-tetraoxadecane or one of their physiologically acceptable salts.

Moreover, it may be inventively preferred to use a p-aminophenol derivative or one of its physiologically acceptable salts as the developer component. Preferred p-aminophenols are especially p-aminophenol, N-methyl-p-aminophenol, 4-amino-3-methyl-phenol, 4-amino-3-fluorophenol, 2-hydroxymethylamino-4-aminophenol, 4-amino-3-hydroxymethylphenol, 4-amino-2-(2-hydroxyethoxy)phenol, 4-amino-2-methylphenol, 4-amino-2-hydroxymethylphenol, 4-amino-2-methoxymethylphenol, 4-amino-2-aminomethylphenol, 4-amino-2-(2-hydroxyethylaminomethyl)phenol, 4-amino-2-(1,2-dihydroxyethyl)phenol, 4-amino-2-fluorophenol, 4-amino-2-chlorophenol, 4-amino-2,6-dichlorophenol, 4-amino-2-(diethylaminomethyl)phenol as well as their physiologically acceptable salts. p-Aminophenol, 4-amino-3-methylphenol, 4-amino-2-aminomethylphenol, 4-amino-2-(1,2-dihydroxyethyl)phenol and 4-amino-2-(diethylaminomethyl)phenol are quite particularly preferred compounds.

Furthermore, the developer component can be chosen from o-aminophenol and its derivatives, such as 2-amino-4-methylphenol, 2-amino-5-methylphenol or 2-amino-4-chlorophenol.

In addition, the developer component can be chosen from heterocyclic developer components such as from pyrimidine derivatives, pyrazole derivatives, pyrazolopyrimidine derivatives and pyrazolopyrazole derivatives and their physiologically acceptable salts. Preferred pyrimidine derivatives are in particular the compounds 2,4,5,6-tetraaminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine and 2,5,6-triaminopyrimidine. Preferred pyrazole derivatives are the compounds that are selected from 4,5-diamino-1-methylpyrazole, 4,5-diamino-1-(2-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-t-butyl-1-methylpyrazole, 4,5-diamino-1-t-butyl-3-methylpyrazole, 4,5-diamino-1-(2-hydroxyethyl)-3-methylpyrazole, 4,5-diamino-1-ethyl-3-methylpyrazole, 4,5-diamino-1-ethyl-3-(4-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2-aminoethyl)amino-1,3-dimethylpyrazole, as well as their physiologically acceptable salts, especially however 4,5-diamino-1-(2-hydroxyethyl)pyrazole. Preferred pyrazolopyrimidines are compounds chosen from pyrazolo[1,5-a]pyrimidine-3,7-diamine, 2,5-dimethyl-pyrazolo[1,5-a]pyrimidine-3,7-diamine, pyrazolo[1,5-a]pyrimidine-3,5-diamine, 2,7-dimethyl-pyrazolo[1,5-a]pyrimidine-3,5-diamine, 3-aminopyrazolo[1,5-a]pyrimidin-7-ol, 3-amino-pyrazolo[1,5-a]pyrimidin-5-ol, 2-(3-aminopyrazolo[1,5-a]pyrimidin-7-ylamino)ethanol, 2-(7-amino-pyrazolo[1,5-a]pyrimidin-3-ylamino)ethanol, 2-[(3-aminopyrazolo[1,5-a]pyrimidin-7-yl)-(2-hydroxy-ethyl)-amino]ethanol, 2-[(7-aminopyrazolo[1,5-a]pyrimidin-3-yl)-(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 3-amino-7-dimethylamino-2,5-dimethylpyrazolo[1,5-a]pyrimidine as well as their physiologically acceptable salts and their tautomeric forms, if a tautomeric equilibrium is present.

A preferred pyrazolopyrazole derivative is 2,3-diamino-6,7-dihydro-1H,5H-pyrazolo[1,2-a]pyrazol-1-one.

Particularly preferred developer components are chosen from at least one compound of p-phenylenediamine, p-toluylenediamine, 2-(2-hydroxy-ethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diamino-2-propanol, bis-(2-hydroxy-5-aminophenyl)methane, 1,3-bis-(2,5-diaminophenoxy)-2-propanol, N,N′-bis-(4-aminophenyl)-1,4-diazacycloheptane, 1,10-bis-(2,5-diaminophenyl)-1,4,7, 10-tetraoxadecane, p-aminophenol, 4-amino-3-methylphenol, 4-amino-2-aminomethylphenol, 4-amino-2-(1,2-dihydroxy-ethyl)phenol and 4-amino-2-(diethyl aminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, as well as the physiologically acceptable salts of these compounds. Quite particularly preferred developer components are p-toluylenediamine, 2-(2-hydroxyethyl)-p-phenylenediamine, 2-methoxy-methyl-p-phenylenediamine, N-(4-amino-3-methylphenyl)-N-[3-(1H-imidazol-1-yl)propyl]amine, and/or 4,5-diamino-1-(2-hydroxyethyl)pyrazole as well as the physiologically acceptable salts of these compounds.

The developer components are preferably used in an amount of 0.0001 to 0.5 wt %, preferably 0.001 to 0.2 wt %, based on the ready-for-use agent.

Coupler components alone, in the context of the oxidative dyeing, do not form any significant coloration; rather, they always need the presence of developer components. Therefore it is inventively preferred that, when using at least one coupler component, at least one developer component is also used. In the context of the invention, coupler components allow at least one substitution of a chemical group of the coupler by the oxidized form of the developer component. A covalent bond is formed between coupler component and developer component. Couplers are preferably cyclic compounds having at least two groups on the ring, chosen from (i) optionally substituted amino groups and/or (ii) hydroxyl groups. If the cyclic compound is a six-membered ring (preferably aromatic), then the stated groups are preferably located ortho or meta to one another,

Inventive coupler components are preferably chosen from at least one compound of: m-aminophenol, o-aminophenol, m-diaminobenzene, o-diaminobenzene and/or their derivatives; naphthalene derivatives with at least one hydroxyl group; di or trihydroxybenzene; pyridine derivatives; pyrimidine derivatives; certain indole derivatives and indoline derivatives; pyrazolone derivatives (e.g., 1-phenyl-3-methylpyrazol-5-one); morpholine derivatives (e.g., 6-hydroxybenzomorpholine or 6-aminobenzomorpholine); quinoxaline derivatives (e.g., 6-methyl-1,2,3,4-tetrahydroquinoxaline), as well as mixtures of two or more compounds from one or more of these classes.

Preferred m-aminophenol coupler components are chosen from at least one compound of 3-aminophenol, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-am ino-4-chloro-2-methylphenol, 5-amino-4-methoxy-2-methylphenol, 5-(2′-hydroxyethyl)-amino-2-methylphenol, 3-diethylaminophenol, N-cyclopentyl-3-aminophenol, 1,3-dihydroxy-5-(methylamino)benzene, 3-ethylamino-4-methylphenol, 2,4-dichloro-3-aminophenol and their physiologically acceptable salts.

Preferred m-diaminophenol coupler components are chosen from at least one compound of m-phenylenediamine, 2-(2,4-diaminophenoxy)ethanol, 1,3-bis(2,4-diaminophenoxy)propane, 1-methoxy-2-amino-4-(2′-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)-amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2′-hydroxyethyl)aminobenzene and their physiologically acceptable salts.

Preferred o-diaminobenzene coupler components are chosen from at least one compound of 3,4-diaminobenzoic acid and 2,3-diamino-1-methylbenzene and their physiologically acceptable salts.

Preferred naphthalene derivatives with at least one hydroxyl group are chosen from at least one compound of 1-naphthol, 2-methyl-1-naphthol, 2-hydroxymethyl-1-naphthol, 2-hydroxyethyl-1-naphthol, 1,3-dihydroxynaphthalene, 1,5-dihydroxynaphthalene, 1,6-dihydroxynaphthalene, 1,7-dihydroxynaphthalene, 1,8-dihydroxynaphthalene, 2,7-dihydroxynaphthalene and 2,3-dihydroxynaphthalene.

Preferred di- or trihydroxybenzenes and their derivatives are chosen from at least one compound of resorcinol, resorcinol monomethyl ether, 2-methylresorcinol, 5-methylresorcinol, 2,5-dimethylresorcinol, 2-chlororesorcinol, 4-chlororesorcinol, pyrogallol and 1,2,4-trihydroxybenzene.

Preferred pyridine derivatives are chosen from at least one compound of 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, 3,5-diamino-2,6-dimethoxypyridine, 3,4-diaminopyridine, 2-(2-methoxyethyl)amino-3-amino-6-methoxypyridine, 2-(4′-methoxyphenyl)amino-3-aminopyridine, and their physiologically acceptable salts.

Preferred pyrimidine derivatives are chosen from at least one compound of 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine and 4,6-dihydroxy-2-methylpyrimidine and their physiologically acceptable salts.

Preferred indole derivatives are chosen from at least one compound of 4-hydroxyindole, 6-hydroxyindole and 7-hydroxyindole and their physiologically acceptable salts.

Preferred indoline derivatives are chosen from at least one compound of 4-hydroxyindoline, 6-hydroxyindoline and 7-hydroxyindoline and their physiologically acceptable salts.

According to the invention, particularly preferred coupler components are chosen from 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)-amino-2-methylphenol, 2,4-dichloro-3-aminophenol, 2-aminophenol, 3-phenylenediamine, 2-(2,4-diaminophenoxy)-ethanol, 1,3-bis(2,4-diaminophenoxy)propane, 1-methoxy-2-amino-4-(2-hydroxyethylamino)-benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl) amino]-2-methoxy-5-methylphenyl}amino)ethanol, 2-({3-[(2-hydroxyethyl)amino]-4,5-dimethylphenyl}amino)ethanol, 2[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-am ino-3-hydroxypyridine, 3-am ino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or mixtures of these compounds or their physiologically acceptable salts. Resorcinol, 2-methylresorcinol, 5-amino-2-methylphenol, 3-aminophenol, 2-(2,4-diaminophenoxy)ethanol, 1,3-bis(2,4-diaminophenoxy)propane, 1-methoxy-2-amino-4-(2′-hydroxyethylamino)benzene, 2-amino-3-hydroxypyridine and 1-naphthol as well as their physiologically acceptable salts are quite particularly preferred.

The coupler components are preferably used in an amount of 0.0001 to 0.5 wt %, preferably 0.001 to 0.2 wt %, based on the ready-for-use agent.

Here, developer and coupler components are generally used in approximately molar amounts relative to one another. Although molar use has also proven to be expedient, a certain excess of individual oxidation dye precursors is not disadvantageous, meaning that developer components and coupler components may be present in a molar ratio of 1 to 0.5 to 1 to 3, particularly 1 to 1 to 1 to 2.

The inventive compositions can further comprise at least one substantive dye. These are dyes that are directly absorbed onto the hair and do not require any oxidative process to develop the color. Substantive dyes can be sub-divided into anionic, cationic and non-ionic substantive dyes. Substantive dyes are usually nitrophenylenediamines, nitroaminophenols, azo dyes, anthraquinones or indophenols. The substantive dyes are preferably used in amounts of 0.0001 to 0.2 wt %, particularly 0.001 to 0.1 wt %, based on total end-use preparation. The total amount of substantive dyes is preferably a maximum of 0.1 wt %.

Preferred anionic substantive dyestuffs are known compounds with the international designations or trade names Acid Yellow 1, Acid Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1 and Acid Black 52, Bromophenol blue and Tetrabromophenol blue. Preferred cationic substantive dyes are cationic triphenylmethane dyes, such as Basic Blue 7, Basic Blue 26, Basic Violet 2 and Basic Violet 14, aromatic systems that are substituted with a quaternary nitrogen group, such as Basic Yellow 57, Basic Red 76, Basic Blue 99, Basic Brown 16 and Basic Brown 17, as well as substantive dyes having a heterocycle that exhibits at least one quaternary nitrogen atom, particularly Basic Yellow 87, Basic Orange 31 and Basic Red 51. The cationic substantive dyes that are commercialized under the trade name Arianor® are quite particularly preferred cationic substantive dyes according to the invention. Non-ionic nitro and quinone dyes and neutral azo dyes are particularly suitable as non-ionic substantive dyes. Preferred non-ionic substantive dyes are those compounds known under the international designations or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 24(4-amino-2-nitrophenyl)aminoFbenzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol. Inventively preferred dye combinations are those having at least the combination of tetrabromophenol blue and Acid Red 92; Tetrabromophenol blue and Acid Red 98; Tetrabromophenol blue and Acid Red 94; Tetrabromophenol blue and Acid Red 87 or Tetrabromophenol blue and Acid Red 51.

Inventive, ready-for-use agents are preferably aqueous, free-flowing preparations. The inventive agents can furthermore comprise all active substances, additives and auxiliaries known for such preparations. The ready-for-use agents from agents (M1) and (M2) here can comprise surface active substances chosen from the above mentioned anionic, non-ionic, zwitterionic and amphoteric surfactants.

Cationic surfactants of the type quaternary ammonium compounds, ester quats and amido amines are inventively preferred in ready-for-use agents. Preferred quaternary ammonium compounds are ammonium halides, especially chlorides and bromides such as alkyltrimethylammonium chlorides, dialkyldimethylammonium chlorides and trialkylmethylammonium chlorides, as well as imidazolium compounds known under the INCI names Quaternium-27 and Quaternium-83. The quaternized protein hydrolyzates illustrate further inventively usable cationic surfactants. Alkylamido amines are normally manufactured by the amidation of natural or synthetic fatty acids and fatty acid fractions with dialkylamino amines, such as stearamidopropyldimethylamine). Likewise preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are marketed, for example, under the trade names Stepantex®, Dehyquart® and Armocare®. The products Armocare® VGH-70, an N,N-bis(2-palmitoyloxyethyl)dimethylammonium chloride, as well as Dehyquart® F-75, Dehyquart® C-4046, Dehyquart® L80 and Dehyquart® AU 35 are examples of such esterquats. Agents used according to the invention preferably comprise cationic surfactants in amounts of 0.05 to 10 wt %, based on total agent. Quantities of 0.1 to 5 wt % are particularly preferred.

In a preferred embodiment, non-ionic, zwitterionic and/or amphoteric surfactants as well as mixtures thereof can be preferred.

According to the invention, the oxidizing agent preparation can be applied to the hair together with a catalyst that activates the oxidation of the dye precursors (e.g., by atmospheric oxygen). Such catalysts include certain enzymes, iodides, quinones or metal ions. Suitable enzymes include peroxidases, which can considerably enhance the effect of small amounts of hydrogen peroxide. An addition of certain metal ions or metal complexes can likewise be preferred. Suitable metal ions include Zn²⁺, Cu²⁺, Fe²⁺, Fe³⁺, Mn²⁺, Mn⁴⁺, Li⁺, Mg²⁺, Ca²⁺and Al³⁺. Zn²⁺, Cu²⁺ and Mn²⁺ are particularly suitable here.

In addition it has proven advantageous when the oxidizing agent preparations comprise at least one stabilizer or complexant. Particularly preferred stabilizers are phenacetin, alkali metal benzoates (sodium benzoate) and salicylic acid.

The addition of complexants is also inventively preferred. Complexants are substances that can complex metal ions. Preferred complexants are chelating agents (i.e., substances that form cyclic compounds with metal ions), wherein a single ligand occupies more than one coordination site on a central atom (i.e., is at least bidentate). Suitable and—in the context of the present invention—preferred chelating agents include polyoxycarboxylic acids, polyamines, ethylenediaminetetraacetic acid (EDTA) and nitrilotriacetic acid (NTA) and hydroxyethane diphosphonic acids and their alkali metal salts. Complexing polymers (i.e., polymers which, either in the main chain itself or laterally thereof, carry functional groups capable of acting as ligands and which react with suitable metal atoms, generally to form chelate complexes) may also be used in accordance with the invention. In this regard, the polymer-bound ligands of the resulting metal complexes can originate from one macromolecule or from various polymer chains. Inventively preferred complexants are nitrogen-containing polycarboxylic acids, especially EDTA, and phosphonates, preferably hydroxyalkane or aminoalkane phosphonates and especially 1,1-hydroxyethane-1,1-diphosphonate (HEDP) or its di or tetrasodium salt and/or ethylenediaminetetramethylene phosphonate (EDTMP) or its hexasodium salt and/or diethylenetriaminepentamethylene phosphonate (DTPMP) or its hepta or octasodium salt.

Further exemplary inventively active products, auxiliaries and additives are non-ionic polymers (such as vinyl pyrrolidinone/vinyl acrylate copolymers, polyvinyl pyrrolidinone and vinyl pyrrolidinone/vinyl acetate copolymers and polysiloxanes); zwitterionic and amphoteric polymers (such as acrylamidopropyltrimethylammonium chloride/acrylate copolymers and octylacrylamide/methyl methacrylate/tert-butylaminoethyl methacrylate/2-hydroxypropyl methacrylate copolymers); anionic polymers (such as polyacrylic acids, vcrosslinked polyacrylic acids, vinyl acetate/crotonic acid copolymers, vinyl pyrrolidinone/vinyl acrylate copolymers, vinyl acetate/butyl maleate/isobornyl acrylate copolymers, methyl vinyl ether/maleic acid anhydride copolymers and acrylic acid/ethyl acrylate/N-tent-butylacrylamid terpolymers); thickeners (such as agar-agar, guar gum, alginates, xanthane gum, gum arabica, karaya gum, locust bean flour, linseed gums, dextrans, cellulose derivatives (e.g., methyl cellulose, hydroxyalkyl cellulose and carboxymethyl cellulose), starch fractions and derivatives like amylose, amylopectin and dextrins, clays such as bentonite or synthetic hydrocolloids such as polyvinyl alcohol; structurants (such as sugar, maleic acid and lactic acid) and texturizers (such as sugar esters, polyol esters or polyalkyl ethers); protein hydrolyzates, particularly those of elastin, collagen, keratin, milk protein, soya protein and wheat protein, their condensation products with fatty acids; perfume oils; cyclodextrins; solvents and solubilisers (such as ethanol, isopropanol, ethylene glycol, propylene glycol, glycerine, dimethylisosorbide and diethylene glycol); defoamers such as silicones; dyestuffs and pigments to color the agent; anti-dandruff active materials (such as Piroctone Olamine, zinc Omadine and Climbazole); photo protective agents (in particular derivatized benzophenones, cinnamic acid derivatives and triazines); active substances (such as allantoin, pyrrolidone carboxylic acids, cholesterol and their salts); other fats and waxes (such as fatty alcohols, beeswax, montan wax and paraffins); swelling and penetration substances (such as glycerine, propylene glycol monoethyl ethers, carbonates); hydrogen carbonates, guanidines, ureas as well as primary, secondary and tertiary phosphates); opacifiers (such as latex, styrene/PVP copolymers and styrene/acrylamide copolymers); pearlizing compositions (such as ethylene glycol mono and distearate as well as PEG-3 distearate); propellants (such as propane-butane mixtures, N₂O, dimethyl ether, CO₂ and air) as well as antioxidants.

One skilled in the art selects these additional materials based on the desired properties of the agent. With regard to further optional ingredients and their amounts used, reference is expressly made to those relevant handbooks known to one skilled in the art, for example, the monograph by K. Schrader, Grundlagen and Rezepturen der Kosmetika, 2nd Ed., Hüthig Buch Verlag, Heidelberg (1989).

Ready-for-use agents of the two-phase agent (M1) and color changing agent (M2) preferably have a pH in the range 6 to 12. Inventively preferred agents exhibit an alkaline pH. Another preferred embodiment of the present invention consists in that the ready for use agent exhibits a pH from 7.0 to 12.0, preferably 8.0 to 11.0. In the context of the present invention, pH values refer to those measured at a temperature of 22 ° C.

The pH is usually adjusted with pH adjustors. The person skilled in cosmetics commonly uses established acidifiers and alkalizers to adjust the pH. Alkalizers that can be used for adjusting the pH are typically chosen from inorganic salts, especially from the alkali metals and alkaline earth metals, organic alkalizers, especially amines, basic amino acids and alkanolamines, and ammonia. Inventively preferred acidifiers are food acids such as citric acid, acetic acid, malic acid or tartaric acid, as well as diluted mineral acids.

Inventively useable organic alkalizers are preferably chosen from alkanolamines from primary, secondary or tertiary amines containing a C₂-C₈ alkyl parent substance carrying at least one hydroxyl group. Inventively quite particularly preferred alkanolamines are chosen from 2-aminoethan-1-ol (monoethanolamine), 2-amino-2-methylpropan-1-ol and 2-amino-2-methyl-propane-1,3-diol. An especially preferred alkanolamine is monoethanolamine. Suitable basic amino acids are lysine, arginine and ornithine. The inventive, inorganic alkalizers are preferably chosen from sodium hydroxide, potassium hydroxide, calcium hydroxide, barium hydroxide, sodium phosphate, potassium phosphate, sodium silicate, potassium silicate, sodium carbonate and potassium carbonate.

Application temperatures can be in a range from 15 to 40 ° C. After a contact time of 2 to 60, preferably 5 to 45 minutes, the blonding agent is removed from the hair by rinsing. There is no need to subsequently wash the hair with a shampoo if a strong surfactant-containing carrier is used.

The inventive agents of the first subject matter of the invention can preferably be used for the oxidative color change of human hair. In this regard, a significant improvement is observed in the surface finish and the gloss of the hair. This improvement can be quantified, for example, by the combability in the wet or dry state after the color change.

Accordingly, a further subject matter of the present invention is the use of an agent of the first subject matter of the invention for improving the surface finish of the fibers when oxidatively color-changing human hair. In the context of this subject matter of the invention, the above enunciated statements apply accordingly.

Depending on the composition of the agents (M1) and (M2), it can be inventively preferred to produce the ready-for-use agent directly prior to use by mixing agent (M1) and agent (M2). This is particularly advantageous for incompatibilities between individual ingredients. Therefore, a preferred presentation form of the ready-for-use agent is a separated packaging unit, wherein each of the agents (M1) and (M2) are separately packaged.

Accordingly, a further subject matter of the present invention is a kit of parts having at least two separate ready-made containers, wherein a first container (C1) comprises a cosmetic agent (M1) according to the first subject matter of the invention as the oxidizing agent preparation and a second container (C2) comprises a color changing preparation (M2) having at least one color changing component in a cosmetic carrier.

In the context of the present invention, a container refers to an encasement that exists in the form of an optionally reusable bottle, a tube, a can, a small bag, a sachet or similar encasement. According to the invention, no limits are set for the encasement material. However, encasements in this regard are preferably made of glass or plastic. An embodiment wherein the encasement of the container that comprises agent (M1) is transparent is particularly preferred in order that the user can visualize the two-phase agent (M1). Accordingly, a preferred embodiment of the inventive kit of parts is one wherein the first container (C1) comprising agent (M1) possesses a transparent packaging, preferably a transparent plastic packaging.

The ready-for-use colorant needs to be formulated and packaged so that firstly, the colorant can be well dispersed onto the keratinic fibers being dyed, and secondly, that it remains in the fibers being dyed during the contact time. For this, it is advantageous if the colorant has a certain viscosity that enables the agent to be applied, but also allows the agent to remain at the place of application. This viscosity can be adjusted by polymeric thickeners in the ready-for-use colorant, wherein this thickener can be present in the color-changing preparation or in the oxidizing agent preparation.

In order to enable good mixing of the color-changing preparation and oxidizing agent preparation, the color-changing preparation and oxidizing agent preparation advantageously exhibit a good free flowability and the increased viscosity of the ready-for-use mixture is then adjusted once the two components have been mixed. One possibility for achieving this aim is the use of polymeric thickeners, whose thickening properties change with the pH. However, highly charged polymers, particularly anionic polymeric thickeners, can lead to problems when manufacturing the oxidizing agent preparation, as such increased addition concentrations of thickeners, particularly for slight variations in pH, can lead to blockages in the production units and equipment, such as metering pumps and valves. Consequently, it is particularly desirable, in addition to raw material savings, to use agents having a lower content of polymeric thickener if this does not impair the viscosity of the ready-for-use mixture.

Accordingly, another object of the present invention is to provide a two-part oxidative colorant for keratinic fibers having good miscibility of the two part-components, but has an adequate viscosity so that the agent can firstly be easily applied, and secondly remain at the active location during application and not flow out of the fibers. Finally, the amount of polymeric thickener in the agent should be reduced so that the above described problems can be minimized or eliminated during the manufacture of the agent.

In an unforeseeable manner, it has now been found that the viscosity of the ready-for-use colorant mixture is made possible by special two-phase cosmetic, oxidative preparations for use in color-changing agents for keratinic fibers, particularly human hair, which comprise an oil in addition to an anionic polymeric thickener. This makes it possible to reduce the added quantity of anionic polymeric thickener without being penalized by the viscosity of the ready-for-use colorant mixture.

Accordingly, a third embodiment of this subject matter of the invention is a kit of parts, wherein the oxidizing agent preparation (M1) in the first phase (I) additionally comprises at least one anionic polymeric thickener chosen from homopolymers or copolymers of acrylic acid and/or methacrylic acid.

As a result, the phase (I) of the oxidizing agent preparation (M1) has at least one anionic, polymeric thickener chosen from homopolymers and copolymers of acrylic acid and/or methacrylic acid. While the oxidizing agent preparation (M1) usually has an acidic pH, the application mixture, however, has an alkaline pH. The polymeric thickener is therefore subjected to a pH change causing deprotonation of the carboxylic acid groups of acrylic acid or methacrylic acid units, and this ionization leads to gel formation and thereby an increase in viscosity.

Besides acrylic acid and methacrylic acid, further examples of anionic monomers that can form the polymeric anionic thickener are crotonic acid, itaconic acid, maleic anhydride and 2-acrylamido-2-methylpropane sulfonic acid. Here, the acidic groups may also be present as the sodium, potassium, ammonium, mono or triethanolammonium salts.

Preferred anionic homopolymers are uncrosslinked and crosslinked polyacrylic acids. Preferred crosslinking agents can be allyl ethers of pentaerythritol, of sucrose and of propylene. Such compounds are commercially available, for example, under the trade name Carbopol®.

Within this embodiment, it can also be preferred to use copolymers of at least one anionic monomer and at least one non-ionic monomer. Regarding the anionic monomers, reference is made to the abovementioned substances.

Preferred non-ionic monomers are acrylamide, methacrylamide, acrylic acid esters, methacrylic acid esters, itaconic acid mono and diesters, vinyl pyrrolidinone, vinyl ethers and vinyl esters.

The inventive oxidizing agent preparation (M1) can preferably additionally comprise at least one anionic polymer or copolymer of acrylic acid and/or methacrylic acid. Preferred known polymers of this type are:
polymers of e.g. at least 10 wt % of lower alkyl esters of acrylic acid, 25 to 70 wt % of methacrylic acid and optionally up to 40 wt % of a further comonomer,
mixed polymers of 50 to 75 wt % ethyl acrylate, 25 to 35 wt % acrylic acid and 0 to 25 wt % of other comonomers. Suitable dispersions of this type are commercially available, e.g. under the trade name Latekoll® D (BASF).
copolymers of 50 to 60 wt % ethyl acrylate, 30 to 40 wt % methacrylic acid and 5 to 15 wt % acrylic acid, crosslinked with ethylene glycol dimethacrylate.

Copolymers of acrylic acid, methacrylic acid or their C₁-C₆ alkyl esters and of the esters of an ethylenically unsaturated acid and an alkoxylated fatty alcohol are also preferred. Suitable ethylenically unsaturated acids include acrylic acid, methacrylic acid and itaconic acid; suitable alkoxylated fatty alcohols include Steareth-20 or Ceteth-20. These types of copolymer are commercialized by the Rohm & Haas Company under the trade name Aculyn® 22 and by the National Starch Company under the trade names Structure® 2001 and Structure® 3001.

Particularly preferred anionic copolymers include copolymers of acrylic acid, methacrylic acid or their C₁-C₆ alkyl esters, as are commercialized under the INCI name Acrylates Copolymers. In this regard, the combination of methacrylic acid and ethyl acrylate as well as optionally crosslinking, multifunctional monomers is preferred. A preferred commercial product for this is, for example, Aculyn® 33 or 33A from the Rohm & Haas Company.

The anionic acrylic acid and/or methacrylic acid polymers or copolymers are preferably present in the inventive agents in an amount of 0.1 to 10 wt %, more preferably 1 to 6 wt % and particularly 2.5 to 4 wt %, based on total weight of the color-changing preparation (M2) and oxidizing agent preparation (M1).

Another embodiment of this subject matter of the invention is one wherein the agent (M2) from container (C2) represents a dye preparation and this color-changing preparation has at least one oxidation dye precursor as the color-changing component.

Moreover, it can be particularly inventively advantageous when the kit-of-parts has at least one additional hair treatment agent, especially a conditioner, in a separate container. Furthermore, the packaging unit can include application aids such as combs, brushes or small brushes, personal protective clothing, especially disposable gloves, as well as an optional instruction manual.

In regard to the preferred embodiments of agents (M1) and (M2), the above embodiments of the preceding subject matters of the invention apply mutatis mutandis.

When using the kit of parts it can be irrelevant whether both phases of agent (M1) are mixed together with briefly vigorous shaking and agent (M2) is added before the phases separate again in order to prepare the ready-for-use color-changing preparation, or whether agent (M2) is added to agent (M1) and then the ready-for-use mixture is produced with thorough mixing.

For improved mixing, it is advantageous if container (C1) comprising the two-phase agent (M1) has a re-closable opening, such as a snap-on or screw closure. This enables the simplified addition of the color-changing agent from container (C2) that itself is preferably in the form of a small bag or sachet in the case of anhydrous, particularly powdery color-changing agents, or in the form of a tube in the case of free flowable color-changing agents. Preferably, the individual preparations are mixed and within a short period of time the ready-for-use agent is applied onto the keratinic fibers.

Accordingly, another subject matter of the invention is a method for changing the color of keratinic fibers, particularly human hair, comprising combining the color-changing preparation (M2) and the oxidizing preparation (M1) from a kit of parts according to the preceding subject matter of the invention in one of the two containers (C1) and (C2), shaking the resealed container, and applying the resulting ready-for-use color changing agent in this container onto the fibers, left on the fibers for a contact period of 5 to 60 minutes and then rinsed out.

For a chromophoric agent, the preferred contact time is 5 to 40 minutes, preferably 10 to 30 minutes. In the case of lightening or bleaching color-changing agents, the preferred contact time is 30 to 60 minutes, preferably 40 to 60 minutes.

A further embodiment of this subject matter of the invention is a method for changing the color of keratinic fibers, particularly human hair, wherein the container (C1) from a kit of parts according to the previous subject matter of the invention is shaken, the resulting mixture of the phases (I) and (II) is then immediately mixed together with a colorant preparation from the container (C2), the resulting, ready for use color changing agent is then applied onto the hair and left for a contact time of 5 to 60 minutes and is then rinsed out.

In the context of this subject matter of the invention, the above enunciated statements apply accordingly.

A further subject matter of the present invention is a ready-for-use agent for the oxidative color changing of keratinic fibers, particularly human hair, wherein the agent is produced immediately prior to use by mixing the components of a kit of parts of the third subject matter of the invention, wherein the viscosity of the agent is 5 to 50 Pa s, preferably 10 to 20 Pa s (Brookfield, 22° C., spindle #5, 4 rpm).

The amount of polymeric, anionic thickener added can be advantageously reduced by combination of oil and anionic polymeric thickener. Firstly, this allows the amounts of raw materials to be reduced, and secondly, problems caused by the high amount of anionic thickener to be minimized in the manufacturing process.

Accordingly a final subject matter of the invention is the use of an oxidizing agent preparation (M1) having two separate phases, wherein the first phase (I) is an aqueous phase having in a cosmetically acceptable carrier at least one chemical oxidizing agent and at least one anionic polymeric thickener chosen from homopolymers or copolymers of acrylic acid and/or methacrylic acid, and wherein the second phase (II) is a hydrophobic phase having at least one oil chosen from paraffin oil or liquid carboxylic acid esters from C₂-C₈ monohydric alkanol with a mono or dicarboxylic acid, for increasing the mix viscosity of a colorant for keratinic fibers, the colorant being produced by mixing a color changing preparation comprising at least one color changing component in a cosmetic carrier and the oxidizing agent preparation (M2).

### EXAMPLES

### 1. Coloration Cream FC Quantities in wt %—

| | FC1 | FC2 |
|---|---|---|
| Lanette D | 6.60 | 6.60 |
| Lorol C12-18 techn. | 2.40 | 2.40 |
| Eumulgin B 2 | 0.60 | 0.60 |
| Eumulgin B 1 | 0.60 | 0.60 |
| LAMESOFT PO 65 | 2.00 | — |
| Akypo Soft 45HP | 10.00 | 10.00 |
| Protelan MST 35 | — | 6.00 |
| Texapon K 14 S Special/0% | 2.80 | 2.80 |
| Produkt W 37194 | 3.75 | 3.75 |
| p-Toluylenediamine sulfate | 0.27 | 2.81 |
| 2,4-Diaminophenoxyethanol 2HCl | — | 0.44 |
| Resorcinol | — | 1.00 |
| 3-Aminophenol | — | 0.20 |
| 5-Amino-2-methylphenol | 0.85 | — |
| 4,5-Diamino-1-(2-hydroxyethyl)pyrazole sulfate | 0.12 | — |
| 4-Chlororesorcinol | 0.15 | — |
| Hydroxyethyl-2-nitro-p-toluidine | 0.13 | — |
| 2.7-Dihydroxynaphthalene | 0.18 | — |
| Sodium hydroxide 45% techn. | 1.60 | — |
| Bis-(5-amino-2-hydroxyphenyl)methane 2HCl | 1.11 | — |
| Potassium hydroxide, aqueous 50% | — | 1.00 |
| Ammonium sulfate techn. pure | 0.50 | — |
| Sodium sulfite | 0.40 | 0.20 |
| HEDP 60% | 0.20 | 0.20 |
| Ascorbic acid E 300 DAB | 0.10 | 0.10 |
| Sodium silicate 40/42 | 0.50 | 0.50 |
| L-Serine | 1.00 | — |
| Glycine | — | 1.00 |
| Taurine | — | 1.00 |
| Alpha-liponic acid | — | 0.20 |
| Litchiderm LS 9704 | — | 1.00 |
| Ammonia 25% | 6.50 | — |
| Monoethanolamine | — | 9.20 |

| | |
|---|---|
| Water, deionized | ad 100 |

Raw materials: Lanette D (INCI name: Cetearyl alcohol; Cognis); Lorol C12-18 techn. (INCI name: Coconut alcohol; Cognis); Eumulgin B2 (INCI name: Ceteareth-20; Cognis); Eumulgin B1 (INCI name: Ceteareth-12; Cognis); Lamesoft PO 65 (ca. 66%, INCI name: Coco-Glucoside, Glyceryl Oleate, Aqua; Cognis); Akypo Soft 45HP (ca. 21%, INCI name: Sodium Laureth-6 Carboxylate, Aqua; KAO); Protelan MST 35 (ca. 35%, INCI name: Sodium Myristoyl Sarconsinate, Sodium Methyl Cocoyl Taurate, Aqua; Zschimmer & Schwarz); Texapon K 14 S Special (ca. 70%, INCI name: Sodium Myreth Sulfate, Aqua; Cognis); Product W 37194 (ca. 20%, INCI name: Acrylamidopropyltrimonium Chloride/Acrylates Copolymer, Aqua; Stockhausen); Litchiderm LS 9704 (INCI name: Butylene Glycol; Lichti Chinensis Pericarp Extract; Laboratoires Serobiologiques).

The fatty base was melted together at 80° C. and dispersed with part of the water. The remaining components of the formulation were then successively incorporated with stirring. Water was then added to make up 100 wt % and the formulation was stirred without heating.

### 2. Developer Preparations EW (Quantities in wt %)—

| Raw material | E1 | E2 | E3 | V1 | V2 | E4 | E5 | E6 |
|---|---|---|---|---|---|---|---|---|
| Sodium hydroxide | 0.73 | 0.73 | 0.73 | 0.73 | 0.73 | 0.73 | 0.73 | 0.73 |
| 45% techn. | | | | | | | | |
| Dipicolinic acid | 0.10 | 0.1 | 0.10 | 0.10 | 0.10 | 0.1 | 0.10 | 0.10 |
| Disodium | 0.03 | 0.03 | 0.03 | 0.03 | 0.03 | 0.03 | 0.03 | 0.03 |
| pyrophosphate | | | | | | | | |
| HEDP 60% | 1.50 | 1.5 | 1.50 | 1.50 | 1.50 | 1.5 | 1.50 | 1.50 |
| Texapon NSO | 2.00 | 2.00 | 2.00 | 2.00 | 2.00 | 2.00 | 2.00 | 2.00 |
| Dow Corning DB | 0.07 | 0.07 | 0.07 | 0.07 | 0.07 | 0.07 | 0.07 | 0.07 |
| 110 A | | | | | | | | |
| Aculyn 33A | 15.00 | 15.00 | 15.00 | 15.00 | 15.00 | 15.00 | 15.00 | 15.00 |
| Hydrogen | 12.00 | 12.00 | 12.00 | 12.00 | 12.00 | 12.00 | 12.00 | 12.00 |
| peroxide 50% | | | | | | | | |
| Cetiol B | 14.29 | — | — | — | — | — | — | — |
| Isopropyl palmitate | — | 14.29 | — | — | — | — | — | — |
| Isopropyl myristate | — | — | 14.29 | — | — | — | 16.66 | 16.66 |
| Liquid paraffin | | | | | — | 16.66 | — | — |
| Water, deionized | ad 100 | ad 100 | ad 100 | ad 100 | ad 100 | ad 100 | ad 100 | ad 100 |

Raw materials: Texapon NSO (ca. 27%, INCI name: Sodium Laureth Sulfate; Cognis); Aculyn 33A (ca. 28%; INCI name: Acrylates Copolymer, Aqua; Rohm & Haas); Dow Corning DB 110 A (INCI name: Dimethicon; Dow Corning).

### 3. Dyeing—

Prior to use, each of the developer solutions were added to the coloration cream FC in a 1:1 weight ratio and mixed together. The freshly produced, ready for use dye (4 g per gram hair, European human hair, Alkinco 6634, #10/2003, A9) was applied. Contact time was 30 minutes at 35° C. for the dye. The strands were then rinsed out with warm water and dried in air. The chestnut brown colored strands exhibited a glossy color and a pleasant feel.

### 4. Wet Combability—

The colored strands were then examined for wet combability versus untreated hair. The strands of hair (European human hair, Alkinco 6634, #10/2003, A9) were washed with 4% sodium laureth sulfate and manually pre-combed three times. Before applying the test formulations, the strands of hair were combed automatically ten times and the required work was determined with the force transfer type WA (Hottinger Baldwin, DE). The measurements were repeated after application of the mixtures V1/FC1, E1/FC1 and E2/FC1. 20 strands of hair were treated and measured for each. The combability value was expressed as the arithmetic mean. The following results were obtained:

| Hair treatment | Combability |
|---|---|
| untreated | — |
| Dyed with FC1 + V1 (not inventive) | +20% |
| Dyed with FC1 + E1 (inventive) | −33% |
| Dyed with FC1 + E2 (inventive) | +1% |

The strands of hair dyed with the inventive agents showed significantly better combability than the strands dyed with comparative agents. The inventive agents therefore significantly improve the keratinic fiber surfaces over corresponding comparative agents.

### 5. Application Mixtures—

The following viscosities of the mixtures were obtained for coloring cream FC2:

| | | | Change in |
|---|---|---|---|
| | | Mixture | viscosity from |
| | | viscosity* | the comparative |
| Colorant | Application mixture | [mPa s] | mixture |
| 1 | FC2 + V2 (non inventive) | 17100 | — |
| 2 | FC2 + E4 (inventive) | 20100 | +17.5% |
| 3 | FC2 + E5 (inventive) | 19900 | +16.4% |
| 4 | FC2 + E6 (inventive) | 17200 | +0.6% |

*measured with a Brookfield-Viskosimeter DV-II + Pro with Spindel #5 at 4 rpm at RT (22° C.) in 590 ml beakers, tall shape.

The inventive, ready for use dyes #2 and #3 exhibit a significantly increase in viscosity versus comparative dye #1. Inventive dye #4 proves that the addition of IPM, even with a reduced addition (13%) of anionic, polymeric thickener, enables constant viscosity of the application mixture to be achieved.

We claim:

1. A cosmetic agent for treating keratinic fibers comprising:
at least two phases that are separated from one another,
wherein the first phase (I) is an aqueous phase having at least one chemical oxidizing agent, and
wherein the second phase (II) is a hydrophobic phase having at least one liquid carboxylic acid ester of a C₂-C₈ monohydric alkanol with a mono or dicarboxylic acid and/or at least one paraffin oil.

2. The agent according to claim 1 wherein the at least one chemical oxidizing agent of phase (I) is chosen from hydrogen peroxide and/or one of its solid addition products on inorganic and/or organic compounds.

3. The agent according to claim 1 wherein the hydrophobic phase (II) comprises a carboxylic acid ester compound having 8 to 22 carbon atoms.

4. The agent according to claim 1 wherein the carboxylic acid ester of the hydrophobic phase (II) is an ester of a C₃-C₄ monohydric alkanol with a mono or dicarboxylic acid.

5. The agent according to claim 1 wherein the carboxylic acid ester of the hydrophobic phase (II) is chosen from isopropyl palmitate, isopropyl myristate and dibutyl adipate.

6. The agent according to claim 1 further comprising non-ionic, anionic, zwitterionic and/or amphoteric surfactants and/or emulsifiers in an amount of 5 wt % or less, based on total weight of the agent.

7. Method of improving the surface finish of keratinic fibers in an oxidative coloration of human hair comprising applying an agent according to claim 1 onto the keratinic fibers.

8. A kit of parts comprising at least two separate ready-made containers, wherein a first container (C1) comprises a cosmetic agent (M2) according to claim 1, and a second container (C2) comprises a color changing preparation (M1) having at least one color changing component in a cosmetic carrier.

9. The kit of parts according to claim 8, wherein the cosmetic agent (M2) in the first phase (I) additionally comprises at least one anionic polymeric thickener chosen from homopolymers and copolymers of acrylic acid and/or methacrylic acid.

10. The kit of parts according to claim 8, wherein the anionic polymeric thickener of the phase (I) is selected from copolymers of at least two different monomers, selected from acrylic acid, methacrylic acid, C₁-C₄ alkyl esters of acrylic acid and/or C₁-C₄ alkyl esters of methacrylic acid

11. The kit of parts according to claim 8, wherein the first container (C1) comprises a transparent packaging.

12. The kit of parts according to claim 8, wherein the color changing component in the color changing preparation (M1) is at least one oxidation dye precursor.

13. Method for changing the color of keratinic fibers comprising:
combining the color changing preparation and the oxidizing agent preparation from the kit of parts according to claim 8 in one of the two containers (C1) and (C2),
resealing and shaking the resealed container,
applying the resulting ready-for-use color changing agent from the container onto the fibers,
leaving the ready-for-use color changing agent on the fibers for a contact period of 5 to 60 minutes, and
rinsing out the ready-for-use color changing agent.

14. Method of preparing a ready-for-use agent for the oxidative color changing of keratinic fibers comprising producing the agent immediately prior to use by mixing the components of a kit of parts according to claim 8, wherein the viscosity of the agent is 5 to 50 Pa s based on Brookfield, 22° C., spindle #5, 4 rpm.
